(19) United States
(12) Patent Application Publication
(10) Pub. No.: US 2026/0287559 A1
(43) Pub. Date: Sep. 24, 2026

(54) HIGH-SPEED TRAILER-INTEGRATED ULTRASONIC INSPECTION SYSTEM WITH MODULAR WHEEL PROBE ASSEMBLY

(71) Applicant: Transtek International LLC, Orlando, FL (US)

(72) Inventors: Fikret Necati CATBAS, Oviedo, FL (US); John Michael Cotter, Dade, FL (US)

(73) Assignee: Transtek International LLC, Orlando, FL (US)

(21) Appl. No.: 19/084,812

(22) Filed: Mar. 20, 2025

(51) Int. Cl. G01N 29/24; G01N 29/22; G01N 29/28; G01N 29/30; G01N 29/42; G01N 33/38

(52) U.S. Cl. CPC G01N 29/2493; G01N 29/225; G01N 29/28; G01N 29/30; G01N 29/42; G01N 33/383; G01N 2291/0232; G01N 2291/106

(57) ABSTRACT

The provided is a wheel probe apparatus for attaching a trailer-based system to enable emission and reception of ultrasonic signals for non-destructive inspection of solid material. The wheel probe apparatus includes a tire assembly having a circumferential surface attached to hub components; a stationary shaft housed within the hub component, the stationary shaft having at least one access port for receiving fluid and electrical signals; at least one bearing mechanism facilitating rotation of the tire assembly around the stationary shaft; at least one ultrasonic transducer module housed within the stationary shaft, configured to alternately transmit and receive ultrasonic waves in a frequency range of 25-500 kHz; and an injector unit for injecting fluid within the wheel probe apparatus. The further provided is a wheel probe assembly for inspecting non-destructive inspection of solid material, including a series of wheel probe apparatuses, a rotating support structure, and a modular connection system.

### TECHNICAL FIELD

The present invention relates to a high-speed, trailer-integrated system for roller- or wheel-based ultrasonic inspection of concrete structures. More specifically, the invention provides a wheel probe apparatus and a modular wheel probe assembly designed for efficient, non-destructive testing of large-scale infrastructure, including roads, bridges, highways, tunnels, and other constructed facilities. The system enables real-time ultrasonic scanning at varying speeds, ensuring accurate defect detection without significant disruption to traffic flow

### BACKGROUND

Ultrasonic measurement systems have been widely used for non-destructive testing of materials, particularly in the inspection of concrete structures such as bridges, roadways, and buildings. These systems are essential for detecting internal defects, such as cracks, delaminations and voids, which can compromise the structural integrity of concrete.

However, existing ultrasonic measurement technologies face several significant limitations, especially when applied to large-scale infrastructure projects. The current industry standard for ultrasonic testing of concrete structures relies heavily on handheld devices. These devices require technicians to physically place the transducer on the surface of the structure, often necessitating lane closures or traffic disruptions. Technicians are exposed to hazardous conditions, especially when working on busy roadways or bridges with high traffic volumes. Handheld devices require manual operation, making the inspection process slow and labor-intensive. Moreover, due to the manual nature of the process, only small sections of a structure can be inspected at a time, leading to incomplete data collection.

In the field, certain ultrasonic measurement devices utilize an ultrasonic transducer array embedded within a wheel. These systems are designed as standalone, handheld units, inherently restricting their operational speed. Wheel probe systems used for the non-destructive testing of concrete are specifically engineered for slow movement, typically operating at walking speeds or significantly below 25 miles per hour. This makes them unsuitable for highway-speed inspections, where traffic flow cannot be significantly disrupted. Other wheel probe systems in the art which are designed for operation up to 25 miles per hour use high-frequency transducers, which are not well-suited for measuring highly attenuative materials like concrete. High-frequency sound waves dissipate quickly in concrete, reducing the accuracy and depth of measurements. Conventional systems are limited by their inability to operate effectively on rough or damaged surfaces, and they often struggle with maintaining consistent data collection due to vibrations and shocks. Additionally, the accuracy of these systems can be compromised by direct transducer-to-transducer transmission, leading to erroneous readings. As infrastructure continues to expand and age, there is a pressing need for innovative solutions that can provide accurate, high-speed inspections without causing disruptions to traffic flow.

For example, U.S. Pat. No. 3,612,920A describes a system with a solid exterior rotating component and a solid interior non-rotating component, separated by a liquid interface. While effective for certain applications, this design lacks the robustness required for inspecting damaged or uneven concrete surfaces. Similarly, U.S. Pat. No. 11,815,495B2 outlines a lower-speed wheel probe system primarily designed for handheld operation, limiting its utility for large-scale inspections at highway speeds.

### SUMMARY

According to the present invention, unlike conventional ultrasonic inspection systems that require slow manual operation, the proposed wheel probe system can be towed behind a trailer at highway speeds (up to 70 miles per hour). This eliminates the need for lane closures or traffic interruptions, significantly improving efficiency in concrete inspections.

The present invention proposes a wheel probe apparatus for attaching a trailer-based system to enable the emission and reception of ultrasonic signals for non-destructive inspection of solid material, the wheel probe apparatus including: a tire assembly having a circumferential surface attached to hub components; a stationary shaft housed within the hub component, the stationary shaft having at least one access port for receiving fluid and electrical signals; at least one bearing mechanism facilitating rotation of the tire assembly around the stationary shaft; at least one ultrasonic transducer module housed within the stationary shaft, configured to alternately transmit and receive ultrasonic waves in a frequency range of 25-500 kHz; and an injector unit for injecting fluid within the wheel probe apparatus.

According to a preferred embodiment of the invention, the wheel probe apparatus uses a flexible tire assembly filled with water, allowing it to operate effectively on rough and damaged road surfaces. Thus, the water-filled tire design absorbs shocks and vibrations, preventing damage to the transducer and ensuring consistent data collection.

According to a preferred embodiment of the invention, the wheel probe apparatus employs at least one transducer per wheel probe, which alternates between transmitting and receiving ultrasonic signals in pulse-echo mode. This configuration ensures that sound waves travel through or reflect off the inspected material before detection, preventing erroneous readings caused by direct transducer-to-transducer transmission. By placing the transducers in separate wheels, the air gap between wheels during operation prevent the sound waves from traveling directly from one transducer to another.

According to a preferred embodiment of the invention, the wheel probe apparatus uses a real-time calibration algorithm based on surface-skimming longitudinal waves to adjust measurements dynamically. High-speed data acquisition (up to 500 pulses per second with typical concrete bridge deck thicknesses) enables the creation of B-scan and C-scan images, providing a comprehensive visualization of subsurface defects. The data interpretation method differentiates between Rayleigh waves, backwall reflections, and other ultrasonic interactions, reducing noise and improving accuracy.

According to a preferred embodiment of the invention, the wheel probe apparatus has an injector unit that delivers fluid (i.e., water) inside the tire. Fluid pressure applied by this injector unit guarantees that the fluid inside the wheel probe will overcome the vacuum created around the stationary shaft during high-speed operation. The injector unit helps to expel air from the inside of the tire by passing it between the stationary shaft and bearing. Air bubbles inside the tire greatly reduce the signal of the sound waves traveling through the liquid filled tires. Fluid injected inside the tire is also expelled through the bearing to cool the bearing during operation. Additional fluid can be sprayed on the exterior of the tire to maintain strong acoustic contact and enhance signal clarity.

According to the present invention, a wheel probe assembly is designed as a modular array of multiple wheel probes, allowing for simultaneous data collection across multiple points of a structure. A rotating support structure enables easy deployment, height adjustment, and storage during transportation. A trailer-mounted configuration allows for easy integration with existing road inspection vehicles and field operations.

The attachable wheel probe assembly is designed to inspect large concrete surfaces at highway speeds, significantly reducing inspection time compared to manual or low-speed systems. Unlike traditional methods that require lane closures, stationary application or specialized slow-moving (hand-held) vehicles, this system allows continuous operation without interrupting traffic.

The present invention also proposes a wheel probe assembly for inspecting non-destructive inspection of solid material, including: a series of wheel probe apparatuses wherein the wheel probe apparatuses are connected with each other; a rotating support structure configured to hold the wheel probe apparatuses; and a modular connection system enabling simultaneous operation and synchronized data collection from multiple wheel probe apparatuses. The proposed wheel probe assembly includes a series of modular wheel probes (each equipped with a transducer) that can be arranged in an array to scan a wide area simultaneously. Multiple wheel probes can be attached in parallel, allowing the system to cover multiple lanes or wider structures in a single pass. This modular design also allows easy maintenance and replacement of individual probes without disrupting the entire system.

The present invention also proposes a system for inspecting non-destructive inspection of solid material, including a trailer configured to support a rotating support structure; a modular wheel probe assembly mounted on the rotating support structure; a fluid management system including a fluid tank, a pump system, and a header for distributing fluid to the wheel probe apparatuses; and an electronic system including: a data storage module, and a remote controller for monitoring and analysis of inspection data. The system having a trailer attached with wheel probe assembly enables high-speed concrete inspection, allowing the system to operate at up to 110 km/h while collecting data. Unlike traditional handheld or slow-moving ultrasonic inspection systems, this setup allows for real-time, non-destructive testing of long concrete structures such as highways, airport runways, and bridges without interrupting traffic.

### BRIEF DESCRIPTION OF THE DRAWINGS

FIG. 1 illustrates a front view of a wheel probe assembly, which includes multiple modular wheel probe apparatuses arranged in a series.

FIG. 2 illustrates a front view of an individual wheel probe apparatus, showing its primary structural components.

FIG. 3 illustrates a side view of an individual wheel probe apparatus shown in FIG. 2.

FIG. 4 illustrates a cross-sectional view of the wheel probe apparatus taken along line A-A of FIG. 3, detailing its internal structure.

FIG. 5 illustrates a top view of a coupling mechanism of the wheel probe apparatus, showing its attachment points.

FIG. 6 illustrates a cross-sectional view of the coupling mechanism along line A-A of FIG. 5, showing its internal configuration.

FIG. 7A illustrates a top view of a stationary shaft of the wheel probe apparatus, showing its alignment features.

FIG. 7B illustrates a cross-sectional view of the stationary shaft taken along line A-A of FIG. 7A.

FIG. 8A illustrates a front view of a hub component of the wheel probe apparatus, showing its external features.

FIG. 8B illustrates a cross-sectional view of the hub component along line A-A of FIG. 8A, depicting the internal arrangement of its components.

FIG. 9 illustrates an exploded view of the components of an individual wheel probe apparatus.

FIG. 10 illustrates a side view of a trailer with an attached wheel probe assembly, which is used to transport and support the wheel probe assembly.

FIG. 11 illustrates a top-down view of a racking system inside the trailer along line A-A of FIG. 10, showing the arrangement of components.

FIG. 12 illustrates a top-down view of a support framework located beneath the trailer along line B-B of FIG. 10, showing its structural layout.

FIG. 13 illustrates a schematic diagram of a system having the wheel probe assembly, showing the interconnections between the major components, including the fluid system, electrical system, and the wheel probe apparatus, demonstrating how they interact.

FIGS. 14A-14D illustrate waves approaching the reflective collimator device wherein FIG. 14A represents an ideal wave, FIG. 14B represents a slightly deflected wave, FIG. 14C represents the deflected waves from erroneous sources and FIG. 14D represents erroneous readings that will bypass the reflections desired of the device, however, they waves will be received exceedingly late and will likely not cause erroneous readings within the time frame considered.

### DETAILED DESCRIPTION OF THE EMBODIMENTS

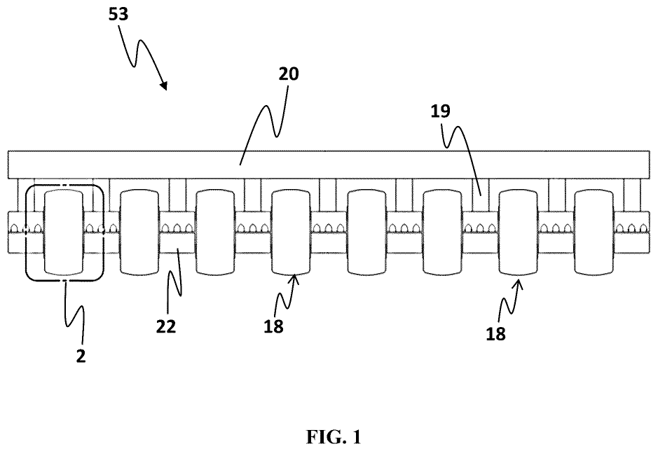

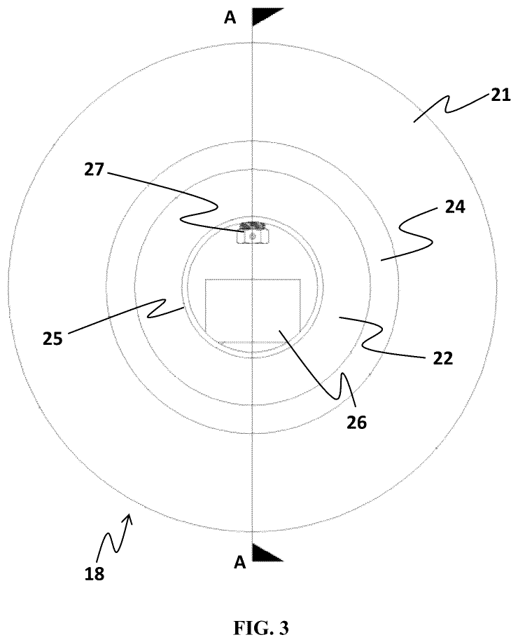

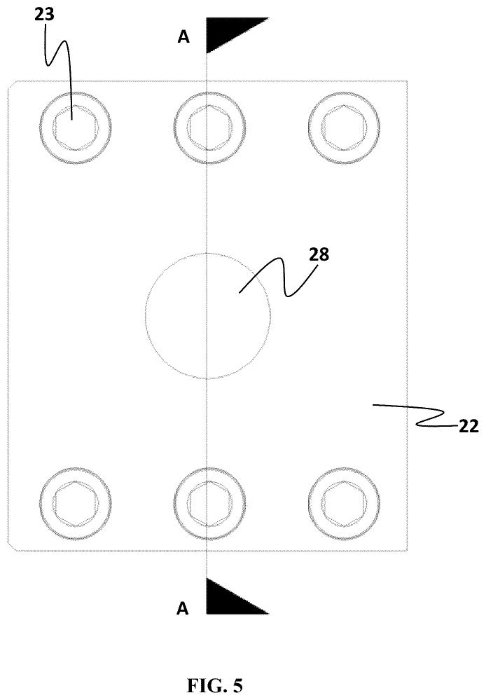

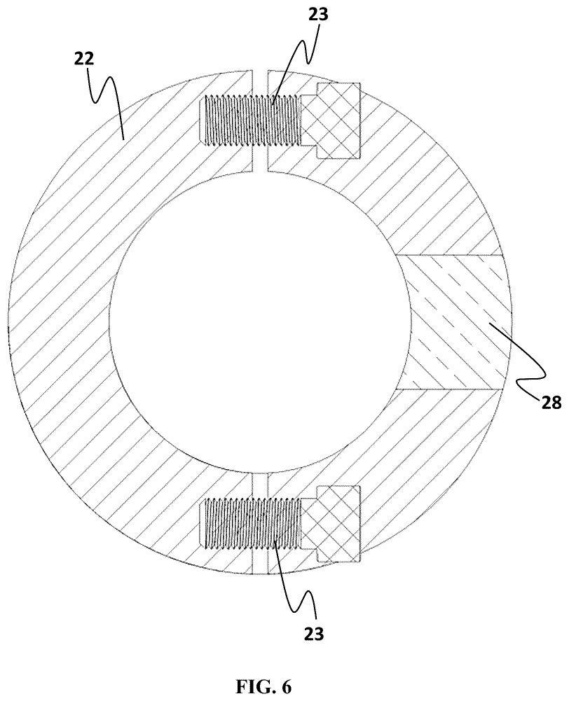

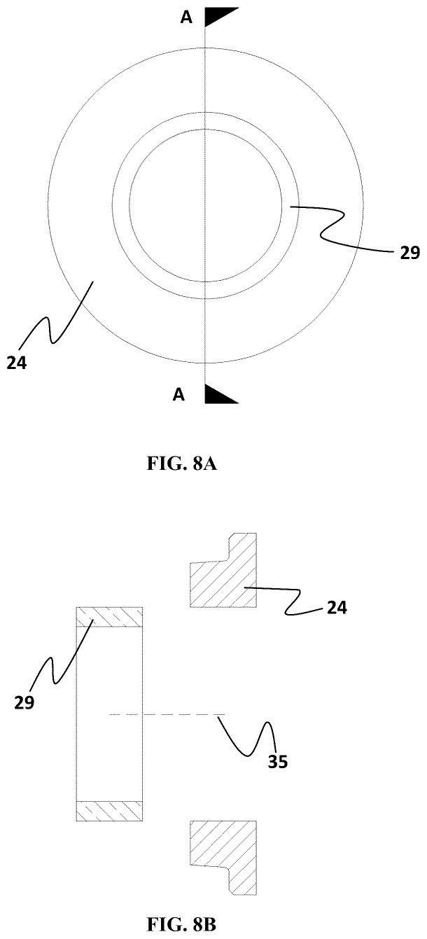

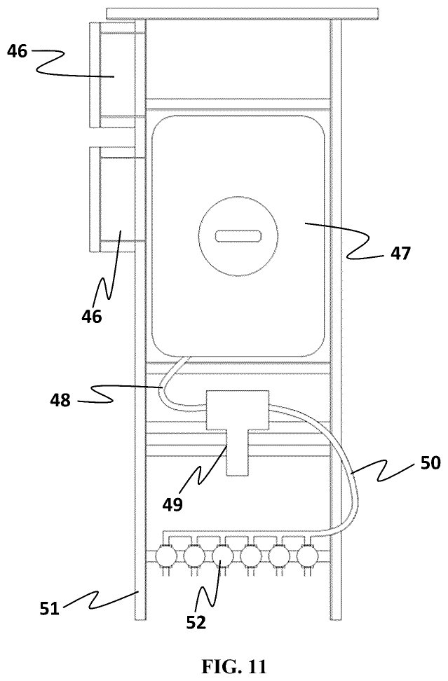

With reference now to the drawings, and in particular to FIG. 1 to FIGS. 14A-14D thereof, a new wheel probe embodying the principles and concepts of an embodiment of the disclosure and generally designed by the reference numeral 18 will be described.

The present invention relates to the wheel probe apparatus (18) for attaching a trailer-based system to enable the emission and reception of ultrasonic signals for non-destructive inspection of solid material. The wheel probe apparatus (18) includes a tire assembly (21) having a circumferential surface attached to oppositely oriented two hub components (24); a stationary shaft (25) housed within the hub component (24), the stationary shaft (25) having at least one access port for receiving fluid (i.e., water) and electrical signals; at least one bearing mechanism (29) facilitating rotation of the tire assembly (21) around the stationary shaft (25); at least one ultrasonic transducer module (26) housed within the stationary shaft (25), configured to alternately transmit and receive ultrasonic waves in a frequency range of 25-500 kHz; and an injector unit (27) for injecting fluid within the wheel probe apparatus (18). The proposed wheel probe apparatus (18) can be mounted on a trailer and can operate at highway speeds, eliminating the need for lane closures.

As depicted in FIG. 1 to FIGS. 14A-14D, a novel wheel probe apparatus (18) and a modular wheel probe assembly (53), which are configured to be attached to an existing trailer (36) are shown. The trailer (36) can facilitate transportation, operation, and testing of the modular wheel probe assembly (53) during testing procedures.

The wheel probe apparatus (18) includes the ultrasonic transducer module (26) configured to measure highly attenuative materials like concrete. According to the present invention, transducer modules (26) are arranged to generate ultrasound in the range of 25-500 kHz and are housed within the stationary shafts (25). Although the transducer modules (26) (as a transducer) are shown as mainly cylindrical devices, they can be employed in any shape, size or direction, and their size, shape or direction may offer certain characteristics pertaining to the direction of the signal's aperture that the transducers will supply. The shape can be tailored to the transmitted signal's particular frequency ranges. For optimal performance, the transducer size and shape as well as arrangements can also be altered to determine whether it will be utilized just for signal emission, signal reception from the test material, or both. In a possible embodiment, the ultrasonic transducer module (26) is housed within the stationary shaft (25), opposite the injector unit (27). The transducer module (26) is securely attached to the shaft using a second sealing interface (31), which ensures the integrity of the watertight environment and prevents leakage of the injected fluid.

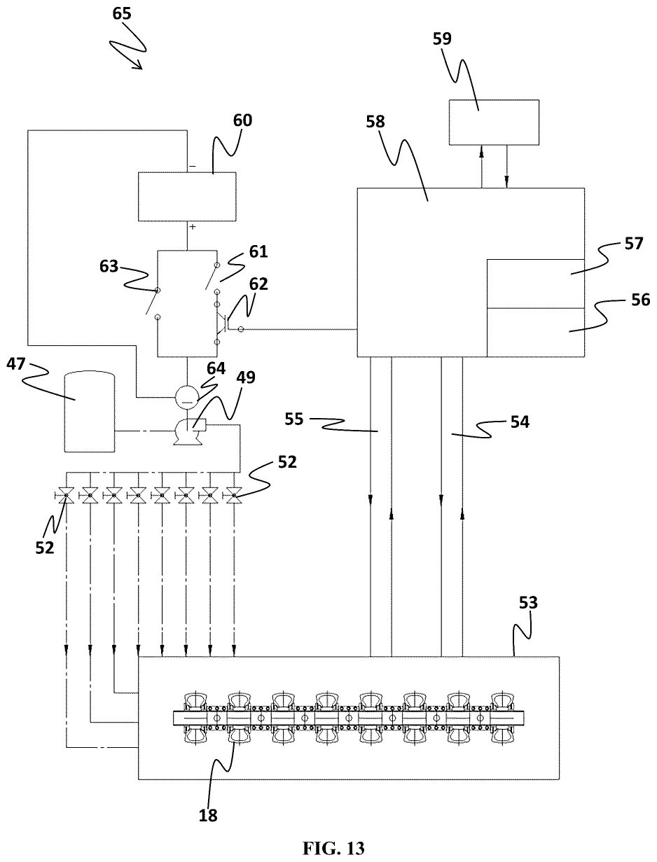

The ultrasonic transducer module (26) alternates between transmitting and receiving ultrasonic sound waves. This system can use water as a coupling medium to enhance signal transmission. These sound waves travel through the solid surface, such as concrete, with some waves propagating along the surface and others penetrating the depth of the material. The high-speed head wave, traveling at longitudinal wave speeds, is used to calibrate the system to the speed of sound in the concrete. Sound waves that travel through the depth of the concrete are analyzed to identify interruptions or deflections caused by defects such as voids, delaminations, and rebar. The voltage signal required to cause the ultrasound waves is generated by the electronic system (58), with the voltage signal transmitted via communication wires (55) to transducer module (26). Data collected by the transducer module (26) is transmitted via communication wires (55) back to the electronic system diagram (58) for processing and storage as shown in FIG. 13.

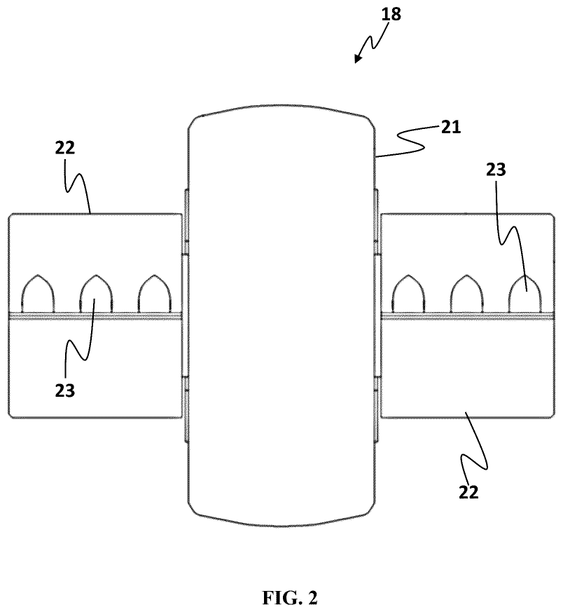

Referring to FIG. 2, the wheel probe apparatus (18) includes a coupling mechanism (22) configured to connect multiple wheel probe apparatuses (18) together. The coupling mechanism (22) utilizes fasteners (23) to clamp securely onto the stationary shaft (25). Each coupling mechanism (22) is provided with an access port (28), enabling the passage of tubing and electrical wiring to establish connections between an electronic system diagram (58) and a header (52), which facilitates fluid and electrical communication between the trailer (36) and the modular wheel probe assembly (53). Moreover, each coupling mechanism (22) can have a coupling support assembly (19) to attach a structural framework (20) formed as a bar, as shown in FIG. 1.

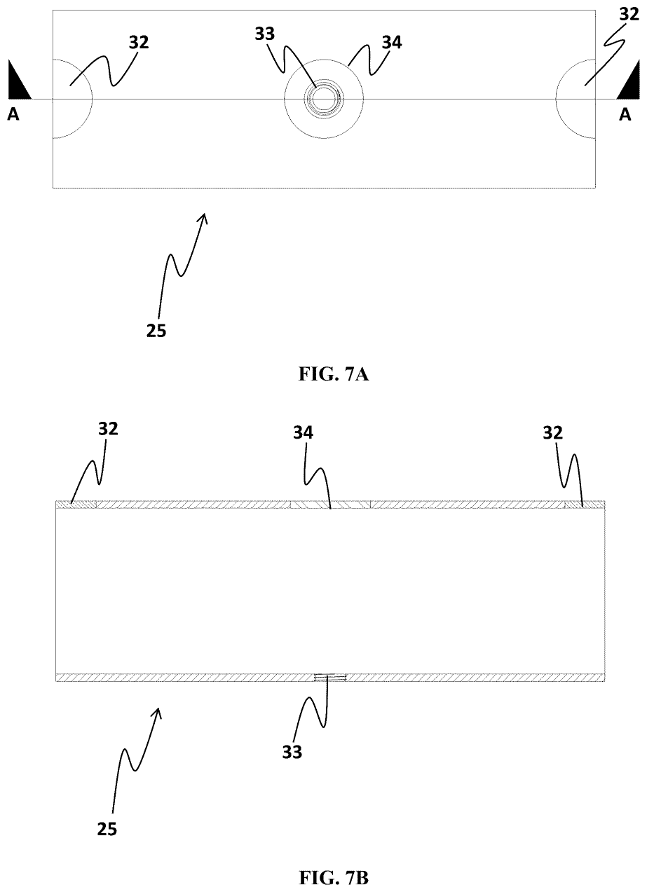

Referring to FIG. 7B, the stationary shaft (25) can be further equipped with at least two semi-circular access ports (32), which are aligned with corresponding access ports on the coupling mechanisms (22). Additionally, the stationary shaft (25) can include a second access port (34), designed to house the ultrasonic transducer module (26), and a threaded hole (33) for securely attaching the injector unit (27) thereof.

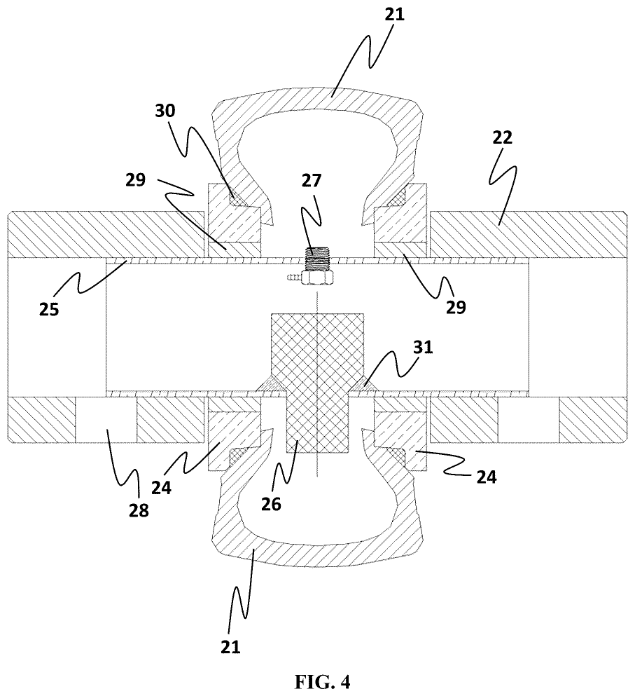

Referring to FIG. 4, the tire assembly (21) can be operatively connected to the hub component (24) through a first sealing interface (30). Each wheel probe apparatus (18) generally includes a tire assembly (21) attached to the hub component (24) via the first sealing interface (30). The first sealing interface (30) ensures a watertight and secure attachment between the tire assembly (21) and the hub component (24). The bearing mechanism (29) can be press-fitted into the hub component (24) to provide rotational support and maintain clearance for injected fluid to expel during operation. The hub component (24) retains the bearing mechanism (29) as illustrated by an indicator line (35), which visually represents the press-fit connection. The hub component (24) can house the bearing mechanism (29), which allows the tire assembly (21) to rotate around the stationary shaft (25). Fluid is mainly injected into the stationary shaft (25) through the injector unit (27) to enhance signal strength and provide cooling. During operation, the bearing mechanism (29) rotates around the stationary shaft (25), which remains fixed relative to the rotational movement of the tire assembly (21).

Advantageously, the injected fluid is intended to leak around the bearing mechanism (29) of the stationary shaft (25), providing both lubrication and cooling. This mechanism prevents excessive wear and potential heat damage to the bearing mechanism (29). In the art, wheel probes tend to expand radially at high rotational speeds. By injecting fluid and allowing it to leak through a controlled interface, this expansion effect is minimized, as the injected fluid will occupy the void created by centrifugal forces. In other words, the injected fluid serves to counteract vacuum formation, which would otherwise occur due to the high-speed rotation of the wheel probe apparatus (18).

When the wheel probe apparatus (18) decelerates, the bearing-wheel hub interface allows the excess fluid to escape naturally, ensuring proper operation without unwanted fluid accumulation. Some parts of the bearing mechanism (29) can be made from bronze due to their cost efficiency. However, more advanced bearing materials, such as lignum vitae or graphite-babbitt alloys, may be employed in future designs, as these materials are optimized for fluid-based cooling applications. The use of a fluid-cooled bearing surface combined with a spray system has the potential to overcome speed limitations in other wheel probe systems, such as those used in the railroad inspection industry. Additionally, the spray system can be configured as a separate unit positioned at the front of the trailer (36) to ensure the surface remains wet by spraying fluid to the road, further enhancing coupling efficiency and overall measurement accuracy. The spray system may be integrated with a sensor-based control mechanism that adjusts the flow rate and spray angle based on real-time road conditions, temperature, or surface dryness. This pre-wetting function optimizes the coupling efficiency between the wheel probe apparatus (18) and the surface, minimizing inconsistencies in signal transmission due to dry or uneven surfaces. Additionally, the spray system may feature a fluid reservoir and pump system mounted on the trailer, supplying a continuous and controlled fluid flow. By actively maintaining surface wetness in advance of the wheel probe apparatus (18), this system enhances ultrasonic wave propagation and ensures accurate, high-speed inspection capabilities.

In a possible embodiment, tire assembly (21) is configured to be filled with water to enhance ultrasonic coupling. This design provides durability and absorbs deflections caused by damaged roadways. The rotation of the tire assembly (21) around the stationary shaft (25) is facilitated by the bearing mechanism (29), allowing for smooth and efficient movement. This may be crucial for the operation of the wheel probe apparatus (18), as it enables the tire assembly (21) to rotate freely while the stationary shaft (25) remains fixed. The tire assembly (21) may be designed to absorb shocks and vibrations, achieved through the use of the flexible tire assembly (21) filled with water. This design may allow the wheel probe apparatus (18) to operate effectively on rough and damaged road surfaces, potentially preventing damage to the transducer and ensuring consistent data collection. The water-filled tire design may also contribute to the prevention of transducer module(s) (26) damage by providing a cushioning effect that absorbs impacts and vibrations. The flexible nature of the tire assembly (21) may allow it to conform to the surface being inspected, enhancing the coupling between the transducer module(s) (26) and the inspected material.

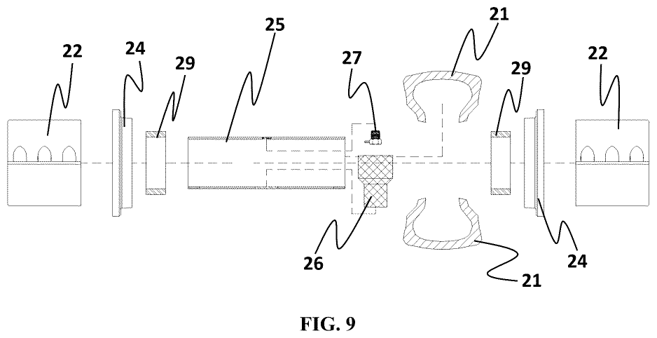

Referring to FIG. 9, fluid (such as water) is introduced into the stationary shaft (25) via the injector unit (27) that is positioned to allow for efficient fluid delivery. The spray system incorporates the injector unit (27) to inject fluid directly into the stationary shaft (25) and bearings, ensuring proper lubrication and cooling. This prevents vacuum effects and enhances the durability of the components. The injector unit (27) is further configured to direct fluid flow through the internal channel within the stationary shaft (25), allowing controlled leakage around the bearing mechanism (29) to provide lubrication and cooling. The fluid that leaks through this interface provides lubricity to the bearing as well as cooling. This prevents excessive wear and potential heat damage of the bearing mechanism (29). When the wheel probe apparatus (18) slows down, the bearing mechanism (29) interface may leak the additional fluid out. This technique of using a fluid-cooled bearing surface and injecting fluid into a wheel probe may alleviate the speed limitations that exist in other wheel probes in the prior art. Moreover, the injector unit (27) formed as a part of the spray unit prevents vacuum formation within the wheel probes apparatus (18) at high speeds.

In another embodiment, the wheel probe apparatus (18) includes an automatic calibration system configured to determine the longitudinal speed of sound in the inspected solid material by analyzing the travel time of ultrasonic waves between known positions of the wheel probe apparatus (18). This automatic calibration system is configured to adjust measurement parameters based on variations in the detected longitudinal wave speed, compensating for ensuring consistent measurement accuracy. This system is configured to determine the longitudinal speed of sound in the inspected material by analyzing the travel time of ultrasonic waves between predefined positions of the wheel probe apparatus (18). The standard delay for the system is defined as a function of electrical signal delay and the time required for ultrasonic waves to propagate through the liquid-filled tire and coupling interface. This auto-calibration is performed in real-time across multiple measurement points, allowing location-specific speed variations to be accounted for during data processing, as the speed of sound is used to calculate and estimate locations of the backwall and defects. The calibration system enables compensation for variations in material properties, such as differences in concrete slump and density, ensuring consistent and reliable ultrasonic readings. The speed of sound can be used as an additional data output from the system as the speed of sound of concrete is an indication of the quality of the concrete.

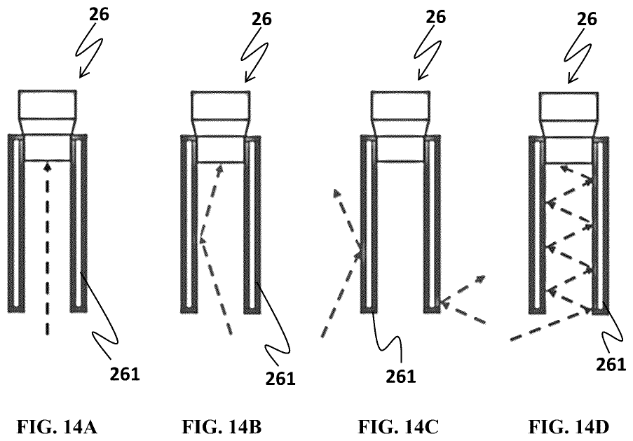

Referring to FIGS. 14A-14D, the ultrasonic transducer module (26) can have an acoustic reflective collimator (261) configured to filter low-frequency ultrasonic waves by attenuating and/or reflecting horizontally propagating sound waves before they reach the transducer module (26). The reflective collimator (261) has a hollow cylindrical body with an internal air gap positioned between an outer structural layer and an inner wave-guiding section, configured to enhance acoustic impedance contrast and selectively reflect undesired ultrasonic wave components while allowing primary measurement signals to pass through. The acoustic reflective collimator (261) is designed to enhance the accuracy of ultrasonic measurements by filtering unwanted low-frequency sound waves. The reflective collimator (261) is a device that modifies the propagation of waves, ensuring directional consistency and preventing signal dispersion. Referring to FIGS. 14A-14D, the reflective collimator (261) is specifically configured to regulate the trajectory of received sound waves and mitigate horizontal wave interference, which commonly affects low-frequency transducers due to their hemispherical wavefront characteristics.

The reflective collimator (261) is constructed using a multi-layered structure including a cylindrical body. The design incorporates an internal air gap positioned between the outer shell and an inner hollow section, forming a high acoustic impedance contrast. This configuration enables efficient reflection of undesired sound waves, preventing contamination of the received ultrasonic signals. The reflective collimator (261) is arranged to operate by leveraging the impedance mismatch, thereby reflecting a substantial portion of errant incident sound waves. The reflection efficiency can be approximated using the known relationship for impedance contrast, ensuring that nearly all non-essential waves are redirected. Experimental verification using 100 kHz transducers demonstrated that sound waves with high-intensity, lower-angle propagation are transmitted efficiently through the reflective collimator (261), whereas waves with extreme angles undergo multiple reflections before exiting, significantly delaying their arrival and minimizing their impact on measurement accuracy.

Referring to FIGS. 14A-14D, the reflective collimator (261) serves to eliminate spurious signals that may arise from environmental noise or unintended reflections. The structural configuration ensures that only waves propagating within a specific angular range reach the transducer, thereby enhancing signal clarity and reducing erroneous readings confirming that the air gap design offers superior performance in applications requiring precision in ultrasonic signal processing. The acoustic reflective collimator's (261) integration within the wheel probe apparatus (18) contributes to enhanced detection reliability, making it particularly effective for inspecting solid materials such as concrete.

The present invention also proposes the wheel probe assembly (53) for non-destructive inspection of solid material, as shown in FIG. 1. The wheel probe assembly (53) includes a series of wheel probe apparatuses (18) in an array and connected with each other; a rotating support structure (45) configured to hold the wheel probe apparatuses (18); and a modular connection system enabling simultaneous operation and synchronized data collection from multiple wheel probe apparatuses (18). The wheel probe assembly (53) is arranged to attach a vehicle (such as a trailer) and is configured to allow for continuous measurement in a rolling system that can function up to 70 miles per hour. This would prevent needing to place technicians on a roadway where cars are traveling and otherwise having to close lanes. This system also allows for readings to be taken while traffic is moving. Position data that can be connected to the received signal data will be provided by the different implementations of the current invention. Position data can include a relative position determined by a starting position specified especially for the construction under analysis and the measured or computed movement from the specified starting point, or an absolute position determined by a GPS or other device capable of defining absolute position. For the transmission of control signals, power, survey data, and other similar information, each connection point of the wheel probe apparatuses (18) may include electrical/signal connections via wire, slip ring contacts, wireless communication, induction contacts, and other means.

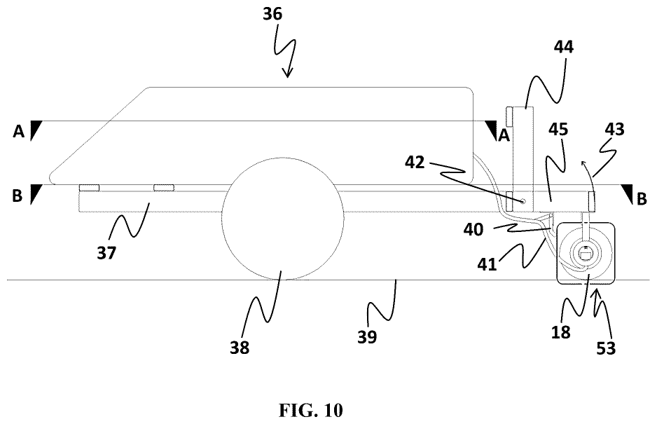

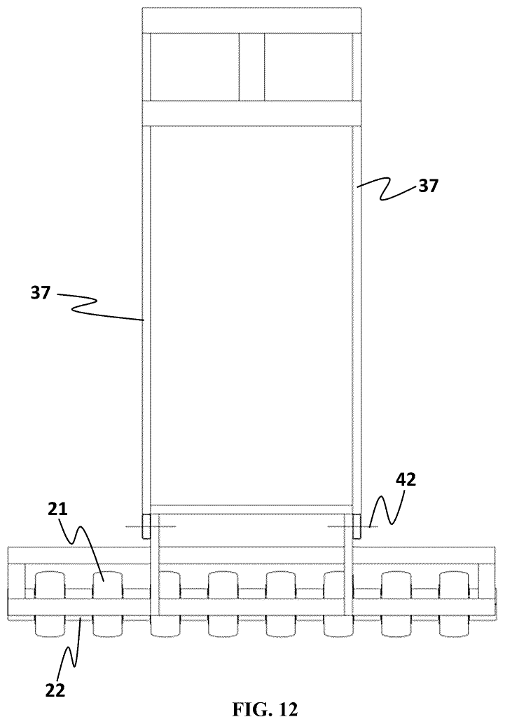

The rotating support structure (45) of the wheel probe assembly (53) is pivotally connected to allow vertical adjustment for transportation. Referring to FIG. 10, a rotational pivot point (42) connects to the rotating support structure (45), which is designed to hold the modular wheel probe assembly (53), including a plurality of individual wheel probe apparatuses (18). The rotational connection allows the rotating support structure to pivot upward, as indicated by a swinging direction (43) in FIG. 12, facilitating ease of transportation and maintenance. Each wheel probe apparatus (18) is configured to operate independently to collect ultrasonic data from different locations on the concrete surface. Moreover, the wheel probe assembly (53) includes an electrical connection system for synchronizing data collection between the wheel probe apparatuses (18).

The present invention also proposes a system (65) for non-destructive inspection of solid material as shown in FIG. 13, the system (65) as a whole includes a trailer (36) configured to support a rotating support structure (45); a modular wheel probe assembly (53) as described above mounted on the rotating support structure (45); a fluid management system including a fluid tank (47), a pump system (49), and the header (52) for distributing fluid to the wheel probe apparatuses (18); and an electronic system including: a data storage module (57), and a remote controller (59) for monitoring and analysis of inspection data. The trailer (36) of the system can be configured to support the modular wheel probe assembly (53) via a support framework of the trailer (37). This support framework includes a vertical extension (44) that is operatively connected to the rotational pivot point (42).

In a possible embodiment, the system (65) includes a manual bypass switch (63) for controlling the pump system (49) during field operations. A manual control switch (61), an electrical relay (62), and the manual bypass switch (63) provide flexibility in operation. The manual control switch (61) and the electrical relay (62) allow for remote activation of the pump motor unit via electronic signals generated by the electronic system diagram. For field testing and manual operation, the manual bypass switch (63) is provided to override the electronic controls and directly operate the pump motor unit and pump system.

The wheel probe system also features an autocalibration algorithm that uses the longitudinal speed of sound to automatically adjust measurements. This algorithm accounts for variations in concrete composition and improves the accuracy of defect detection.

The trailer (36) of the system (65) is modified to house a support framework (37), which includes the vertical extension (44) and the rotational pivot point (42). The pivot point (42) connects to the rotating support structure (45) that holds the modular wheel probe assemblies (53). Multiple wheel probe apparatuses (18) are mounted on the rotating support structure (45) connected to the trailer (36). The modular design enables simultaneous data collection from multiple locations on the concrete surface. During operation, the support structure can pivot upward for transportation and maintenance, as indicated by the swinging direction (43) shown in FIG. 10.

The system (65) includes a calibration module configured to adjust transducer parameters based on real-time environmental conditions. The system (65) can also include a pressure regulation system configured to control the fluid flow rate within the stationary shafts (25) of the wheel probe apparatuses (18) to optimize acoustic coupling and prevent excess fluid loss. A ground surface (39) shown in FIG. 10 provides foundational support for both a trailer wheel assembly (38) and the wheel probe apparatuses (18). A supply line (41) is operatively coupled with the system (65) to facilitate the transfer of fluid and electrical signals. This supply line enables the transmission of high-voltage pulses and data, as well as the delivery of fluid to a fluid spray line (40) and the injector units (27).

Within the trailer (36), a rack structure (51) can house the system's electronic and fluid management components. This includes at least one battery compartment (46) for accommodating at least one battery (56) and a pump battery (60). The fluid tank (47), supported by the rack structure and the trailer (36), supplies fluid through a first tubing (48) to the pump system (49). The pump system (49) delivers fluid through a second tubing (50) to the header (52), which is configured with piping and valves to distribute fluid efficiently to various components. The fluid management system can include the fluid tank (47), a pump system (49), and the header (52) for distributing fluid to the tires and fluid sprays. Fluid is delivered through the first tubing (48) and the second tubing (50), ensuring optimal signal quality and cooling of the bearings. The fluid management system can be powered by the pump battery (60) and a pump motor unit (64). The pump battery (60) powers the pump motor unit (64), which drives the pump system (49). Within the trailer (36), the rack structure (51) is designed to house the system's electronic and fluid management components. This includes at least one battery compartment (46) for accommodating at least one battery (56) and a pump battery (60), which powers the fluid management system. The fluid management system is responsible for storing, pumping, distributing, and regulating the flow of fluid to key components, ensuring optimal ultrasonic coupling, bearing lubrication, and cooling. The fluid management system is powered by a dedicated pump battery (60). The pump motor unit (64), driven by the pump battery (60), provides the necessary power to operate the pump system (49). Sensors and automated valves may be included to dynamically adjust fluid flow based on road conditions, wheel probe operation, and ultrasonic signal quality.

The electronic system is powered by a self-contained battery (56) housed in the battery compartment (46). Data is stored in the data storage module (57) and can be accessed remotely using the remote controller (59). The remote controller (59) and the data storage module (57) enable real-time monitoring and analysis without direct access to the trailer (36). the remote controller (59) that allows for wireless communication with the electronic system diagram, enabling remote monitoring and control without direct access to the trailer (36).

The invention further includes the electronic system diagram (58), which receives input from a signal output (54) providing rotational speed data from the wheel probe apparatuses. Electrical pulses are transmitted and received via communication wires (55) to activate the ultrasonic transducer modules (26). These modules are configured to alternately transmit and receive ultrasonic waves, as dictated by the control logic within the electronic system diagram. The data storage module (57) is integrated into the system to collect and store operational data, which can later be retrieved for analysis. The proposed system can operate at highway speeds, collecting data with a pulse rate of up to 500 pulses per second, enabling rapid and comprehensive inspections. Collected data can be processed to generate B-scan and C-scan images, identifying defects such as voids, delaminations, spalling, and rebar locations.

### REFERENCE NUMBERS

| | |
|---|---|
| 18. Wheel probe apparatus | 42. Rotational pivot point |
| 19. Coupling support assembly | 43. Swinging direction |
| 20. Structural framework | 44. Vertical extension |
| 21. Tire assembly | 45. Rotating support structure |
| 22. Coupling mechanism | 46. Battery compartment |
| 23. Fastener | 47. Fluid tank |
| 24. Hub component | 48. First tubing |
| 25. Stationary shaft | 49. Pump system |
| 26. Transducer module | 50. Second tubing |
| 261. Reflective collimator | 51. Rack structure |
| 27. Injector unit | 52. Header |
| 28. Access port | 53. Wheel probe assembly |
| 29. Bearing mechanism | 54. Signal output |
| 30. First sealing interface | 55. Communication wire |
| 31. Second sealing interface | 56. Battery |
| 32. First access Port | 57. Data storage module |
| 33. Threaded Hole | 58. Electronic system diagram |
| 34. Second access Port | 59. Remote controller |
| 35. Indicator line | 60. Pump battery |
| 36. Trailer | 61. Manual control switch |
| 37. Support framework | 62. Electrical relay |
| 38. Trailer wheel assembly | 63. Manual bypass switch |
| 39. Ground surface | 64. Pump motor unit |
| 40. Fluid spray line | 65. System |
| 41. Supply line | |

What is claimed is:

1. A wheel probe apparatus for enabling emission and reception of ultrasonic signals for non-destructive inspection of a solid material, comprising:
a tire assembly having a circumferential surface attached to a hub component;
a stationary shaft housed within the hub component, the stationary shaft having at least one access port for receiving a fluid and electrical signals;
at least one bearing mechanism facilitating rotation of the tire assembly around the stationary shaft;
at least one ultrasonic transducer module housed within the stationary shaft, wherein the at least one ultrasonic transducer module is configured to alternately transmit and receive ultrasonic waves in a frequency range of 25-500 kHz; and
an injector unit for injecting the fluid within the wheel probe apparatus.

2. The wheel probe apparatus according to claim 1, wherein the tire assembly is configured to be filled with water to enhance ultrasonic coupling.

3. The wheel probe apparatus according to claim 1, wherein the injector unit is further configured to direct fluid flow through an internal channel within the stationary shaft, allowing controlled leakage around the at least one bearing mechanism to provide lubrication and cooling.

4. The wheel probe apparatus according to claim 1, further comprising an automatic calibration system configured to determine a longitudinal speed of sound in an inspected solid material by analyzing a travel time of ultrasonic waves between known positions of the wheel probe apparatus.

5. The wheel probe apparatus according to claim 4, wherein the automatic calibration system is configured to adjust measurement parameters based on variations in a detected longitudinal wave speed, compensating for ensuring consistent measurement accuracy.

6. The wheel probe apparatus according to claim 1, further comprising an acoustic reflective collimator configured to filter low-frequency ultrasonic waves by attenuating horizontally propagating sound waves before the low-frequency ultrasonic waves reach the at least one ultrasonic transducer module.

7. The wheel probe apparatus according to claim 6, wherein the acoustic reflective collimator comprises a hollow cylindrical body with an internal air gap positioned between an outer structural layer and an inner wave-guiding section, and the acoustic reflective collimator is configured to enhance acoustic impedance contrast and selectively reflect undesired ultrasonic wave components while allowing primary measurement signals to pass through.

8. The wheel probe apparatus according to claim 1, wherein the wheel probe apparatus comprises a coupling mechanism configured to connect a plurality of wheel probe apparatuses together.

9. The wheel probe apparatus according to claim 1, wherein the injector unit is provided within the stationary shaft and is configured to deliver the fluid into the stationary shaft wherein the injector unit is configured to be in communication with a spray system for receiving the fluid.

10. The wheel probe apparatus according to claim 1, wherein the wheel probe apparatus is configured to attach a trailer-based system.

11. A wheel probe assembly for non-destructive inspection of a solid material, comprising:
a plurality of wheel probe apparatuses according to claim 1, wherein the plurality of wheel probe apparatuses are connected with each other;
a rotating support structure configured to hold the plurality of wheel probe apparatuses; and
a modular connection system enabling simultaneous operation and synchronized data collection from the plurality of wheel probe apparatuses.

12. The wheel probe assembly according to claim 11, wherein the rotating support structure is pivotally connected to allow vertical adjustment for transportation.

13. The wheel probe assembly according to claim 11, wherein each of the plurality of wheel probe apparatus is configured to operate independently to collect ultrasonic data from different locations on the solid material.

14. The wheel probe assembly according to claim 11, wherein the wheel probe assembly comprises an electrical connection system for synchronizing data collection between the plurality of wheel probe apparatuses.

15. A system for inspecting non-destructive inspection of a solid material, comprising:
a trailer configured to support the rotating support structure;
the wheel probe assembly according to claim 11 mounted on the rotating support structure;
a fluid management system comprising a fluid tank, a pump system, and a header for distributing the fluid to each of the plurality of wheel probe apparatuses; and
an electronic system comprising a data storage module, and a remote controller for monitoring and analysis of inspection data.

16. The system according to claim 15, wherein the trailer comprises a support framework with a vertical extension and a rotational pivot point.

17. The system according to claim 15, wherein the system comprises a calibration module configured to adjust transducer parameters based on real-time environmental conditions.

18. The system according to claim 15, wherein the system comprises a spray system in fluid communication with the injector unit for providing the fluid to the injector unit, wherein the injector unit is located within the stationary shaft for delivering the fluid into the stationary shaft.

19. The system according to claim 18, wherein the spray system is formed as a separate unit and is configured to move ahead of the trailer.

20. The system according to claim 19, further comprising a pressure regulation system configured to control a flow rate of the fluid coming from the spray system, within the stationary shaft of the wheel probe apparatus.
